(19) United States
(12) Patent Application Publication
(10) Pub. No.: US 2006/0127329 A1
(43) Pub. Date: Jun. 15, 2006

(54) Tartar control oral care composition containing extract of magnolia

(75) Inventors: Tao Xu, East Brunswick, NJ (US); Susan Herles, Flemington, NJ (US); James R. Brown, Edison, NJ (US); Virginia Barnes, Annandale, NJ (US); Rose Richter, Somerville, NJ (US); Michael Prencipe, West Windsor, NJ (US)

Correspondence Address: COLGATE-PALMOLIVE COMPANY, 909 RIVER ROAD, PISCATAWAY, NJ, 08855, US

(73) Assignee: Colgate-Palmolive Company

(21) Appl. No.: 11/256,789

(22) Filed: Oct. 24, 2005

Related U.S. Application Data
(60) Provisional application No. 60/635,352, filed on Dec. 10, 2004.

(51) Int. Cl. A61K 36/575; A61K 8/46; A61K 8/97

(52) U.S. Cl. 424/57; 424/58; 424/769

(57) ABSTRACT

A stable and efficacious antiplaque, anticalculus, and antigingivitis oral composition is provided having an antibacterial ingredient comprising one or more active compounds found in an extract of magnolia combined with an anticalculus system comprising tetrasodium pyrophosphate (TSPP) about 1 to about 2.5% and sodium tripolyphosphate (STPP) about 1 to about 10%. The oral composition can be in the form of a mouth rinse or dentifrice, including toothpaste, gels, powders, as well as confectionaries, lozenges, and the like. Methods of making and using the oral composition are also provided.

### CROSS-REFERENCE TO RELATED APPLICATIONS

This application claims the benefit of U.S. provisional patent application Ser. No. 60/635,352, filed Dec. 10, 2004, the contents of which are incorporated herein by reference.

### BACKGROUND OF THE INVENTION

Dental plaque is a soft deposit which forms on the surfaces of teeth. Dental plaque is generally believed to be formed as a byproduct of bacterial growth and comprises a dense microbial layer consisting of a mass of microorganisms embedded in a polysaccharide matrix. Plaque tenaciously adheres to the surfaces of teeth, especially along irregular and rough surfaces, and is typically found at the gingival margin, in cracks in the enamel, and on the surface of built-up dental calculus.

Gingivitis is the inflammation or infection of the gums and the alveolar bones that support the teeth. Gingivitis is generally believed to be caused by bacteria in the mouth (particularly the bacteria instigated in plaque formation) and the toxins formed as byproducts from the bacteria. Periodontitis is generally believed to occur where unremoved plaque hardens into calculus (tartar) which effects the periodontal ligaments. Periodontitis is a progressively worsened state of disease as compared to gingivitis. As plaque and calculus continue to build up, the gums begin to recede from the teeth and pockets form therebetween, which ultimately may result in destruction of the bone and periodontal ligament. These reactions lead to the destruction of the supporting structure, continued infection, and potentially the subsequent loss of teeth.

The plaque formed along the tooth surfaces thus provides a locus for calculus or tartar formation. Dental calculus, or tartar, is a hard mineralized solid formed on the teeth when crystals of calcium phosphate are deposited in the pellicle and extracellular matrix of the dental plaque and become crystalline hydroxyapatite, sufficiently closely packed together for the aggregates to become resistant to deformation. Regular brushing aids in preventing a rapid build-up of these deposits, but even regular brushing is not sufficient to remove all of the calculus deposits which adhere to the teeth. While there is no complete agreement on the route by which precipitated calcium and orthophosphate ultimately become the crystalline material called hydroxyapatite (HAP), it is generally agreed that at higher saturations (above the critical saturation limit) the precursor to crystalline HAP is an amorphous or microcrystalline calcium phosphate. “Amorphous calcium phosphate” although related to hydroxyapatite differs from it in atomic structure, particle morphology, and stoichiometry. Agents which effectively interfere with crystalline growth of HAP will be effective as anticalculus agents. One suggested mechanism by which many anticalculus agents inhibit calculus formation involves an increase of the activation energy barrier thus inhibiting the transformation of precursor amorphous calcium phosphate to HAP. Studies have shown that there is a good correlation between the ability of a compound to prevent HAP crystalline growth in vitro and its ability to prevent calcification in vivo, provided that the compound is stable in and inert to other components in an oral care composition and to saliva in the oral cavity.

Thus, it is desirable to have an oral care composition that combats plaque by antibacterial activity and further controls and prevents calculus formation. It is difficult to predict the antiplaque efficacy of antibacterial compounds when incorporated in a delivery vehicle and particularly in oral compositions having other active ingredients, such as tartar control agents. For example, cationic antibacterial compounds such as chlorhexidine, benzothonium chloride and cetyl pyridinium chloride, and nonionic antibacterial compounds such as halogenated hydroxydiphenyl ethers, including Triclosan, have generally been found to be ineffective when anionic surfactants or anionic active ingredients (tartar control phosphates, for example) are included in the composition. There is often a negative interaction between an antibacterial agent with other active ingredients or other components in a delivery vehicle of an oral care composition that reduces the effective performance of such oral compositions, including toothpaste and mouthrinses. This is especially true for many tartar control systems, because generally they are anionic compounds, and have a propensity to negatively interact with antibacterial agents, which reduces the efficacy and/or bioavailability of the antibacterial and/or anticalculus active ingredients.

Notwithstanding the efficacy of certain antibacterial agents, there is a continuing interest in the oral care field for oral care compositions which improve the treatment of both plaque and tartar formation. Thus, there is a need for a highly effective antibacterial, antiplaque and anticalculus oral care composition to prevent or diminish oral care diseases, without suffering from negative interaction between the ingredients.

### BRIEF SUMMARY OF THE INVENTION

The present invention generally relates to an antiplaque and anticalculus oral care composition containing a stable and efficacious combination of an antibacterial agent comprising an extract of Magnolia officinalis and an anticalculus system.

In an embodiment of the present invention, a stable antiplaque, anticalculus, and antigingivitis oral composition is provided comprising a safe and effective amount of an antibacterial ingredient comprising at least one active compound from an extract of magnolia, as well as a safe and effective amount of an anticalculus system comprising tetrasodium pyrophosphate (TSPP) and sodium tripolyphosphate (STPP). The TSPP is present about 0.5 to about 2.5% and the STPP is present about 1 to about 10% based on the total weight of the composition. The oral composition also comprises an orally acceptable carrier.

In another embodiment of the present invention, the oral composition is stable and efficacious as an antiplaque, anticalculus, and antigingivitis oral composition, and comprises a safe and effective amount of an antibacterial ingredient comprising at least one active compound from an extract of magnolia at a concentration of about 0.001 to about 10% of the composition. The oral composition also comprises a safe and effective amount of an anticalculus system consisting essentially of tetrasodium pyrophosphate (TSPP), sodium tripolyphosphate (STPP), and a copolymer of maleic anhydride and methyl vinyl ether. The TSPP is present about 0.5 to about 2.5%, the STPP is present about 1 to about 10%, and the copolymer is present at a concentration of about 0.5 to about 1.5% based on the total weight of the composition. The oral composition comprises an orally acceptable carrier.

In an embodiment of the present invention, a method of treating plaque and calculus on an oral surface of a mammalian subject is provided, where the method comprises preparing an oral care composition comprising an orally acceptable carrier; a safe and effective amount of an antibacterial ingredient comprising at least one active compound from an extract of magnolia; a safe and effective amount of an anticalculus system comprising tetrasodium pyrophosphate (TSPP) and sodium tripolyphosphate (STPP). The TSPP is present about 0.5 to about 2.5% and the STPP is present about 1 to about 10% based on the total weight of the composition. The method comprises contacting the oral care composition with the oral surface to thereby reduce both plaque formation and calculus formation.

In another embodiment, a stable antiplaque, anticalculus, and antigingivitis oral composition comprises a safe and effective amount of an antibacterial ingredient comprising at least one active compound from an extract of magnolia. The oral composition also comprises a safe and effective amount of an anticalculus system comprising tetrasodium pyrophosphate (TSPP) and sodium tripolyphosphate (STPP). A ratio of TSPP to STPP ranges about 1:2 to about 1:4 in the anticalculus system. The oral composition also comprises an orally acceptable carrier.

It has been discovered that compositions and methods of this invention afford advantages over antibacterial and antiplaque compositions among those known in the art. Such advantages include providing an oral care composition that is stable and highly effective both as an antibacterial/antiplaque treatment as well as an anticalculus treatment for preventing and/or ameliorating gingivitis and/or periodontitis. Further, the oral composition comprises an antibacterial active ingredient that is natural and derived from a botanical source. Further uses, benefits and embodiments of the present invention are apparent from the description set forth herein.

### DETAILED DESCRIPTION OF THE INVENTION

The present invention provides a highly effective oral composition for both preventing plaque and tartar formation on the surfaces of teeth in the oral cavity, which promotes overall oral and system health, including preventing one or more of gingivitis, periodontitis, and halitosis.

The compositions of the present invention comprise at least one active compound found in an extract of magnolia. As referred to here, such an “extract” of magnolia is an extract from dried cortex, or bark, of a plant from the Magnoliaceae family, such as Magnolia officinalis, (hereinafter “magnolia”) or a synthetic or semi-synthetic equivalent of such an extract or an active component thereof. In certain embodiments of the present invention, the antibacterial ingredient in the active composition comprises one or more active compounds that have been isolated from an extract of magnolia. In other embodiments, the antibacterial ingredient comprises an extract of magnolia. Preferably, extracts of Magnolia Cortex (the bark of Magnolia officinalis) contain active compounds including: magnolol, honokiol, tetrahydromagnolol, and tetrahydrohonokiol, which have demonstrated bactericidal properties against representative oral bacteria S. mutans, F. nucleatum, V. parvula, A. naslundii, P. gingivitis in the in vitro test Minimal Inhibitory Concentration (MIC). It should be noted that any plant from the Magnoliaceae family is suitable for the present invention and may be used in alternate embodiments, preferably such that the extract comprises an antimicrobially effective concentration of a compound selected from the group consisting of magnolol, honokiol, tetrahydromagnolol, tetrahydrohonokiol, and mixtures thereof.

As used herein, “extracting” or “extraction” of a solid or liquid material means contacting the material with an appropriate material, such as a solvent to remove the substance(s) desired to be extracted from the material. Such an extraction may be carried out by conventional means known to one of skill in the art, for example, by using an extraction apparatus, such as a Soxhlet apparatus, which retains the solid material in a holder and allows the solvent to flow through the material; or by blending the solvent and material together and then separating the liquid and solid phases or two immiscible liquid phases, such as by filtration or by settling and decanting.

In one embodiment of the present invention, the magnolia extract is isolated by supercritical fluid extraction (SFE) using carbon dioxide (CO₂).

In various embodiments, it is preferred that the active antibacterial ingredient comprises either magnolol, honokiol, or both. Magnolol and honokiol are non-ionic hydroxybiphenyl compounds, the structures of which are represented as follows:

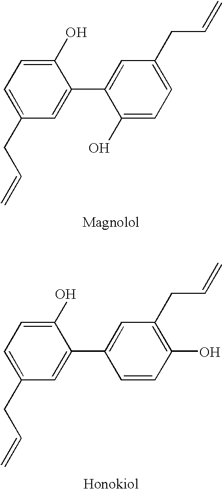

Additionally, tetrahydromagnolol and tetrahydrohonokiol are hydrogenated analogs of magnolol and honokiol often found in relatively small concentrations in the extracts of magnolia, and as such may be included in the antibacterial ingredient.

In various embodiments of the present invention, the oral care composition shall include a safe and effective amount of at least once active compound from the magnolia extract. Accordingly, the amount of compound is to have the desired therapeutic or prophylactic effect in the human or lower animal subject to whom the active is administered, without undue adverse side effects (such as toxicity, irritation, or allergic response), commensurate with a reasonable benefit/risk ratio when used in the manner of this invention. The specific safe and effective amount of the compound will vary with such factors as the particular condition being treated, the physical condition of the subject, the nature of concurrent therapy (if any), the specific compound used, the specific dosage form, the carrier employed, and the desired dosage regimen.

Additionally, the concentration the compound from a magnolia extract in the oral care composition will vary depending on delivery form, dosage regimen, end benefits, pathology, and/or the individual therapeutic requirements of the subject(s) to whom it is admitted depends upon the relative concentration of the active compounds in the extract, and as such, it is contemplated that the amount of magnolia extract present may vary as recognized by one of skill in the art. Additionally, the concentration of the active ingredients is typically dependent upon the form of the oral composition. For example, mouthrinses typically have a relatively low concentration of an active ingredient, as where dentifrices, gels, or toothpowders have a higher concentration to achieve the same delivered dosage based on ease of dispersion. Likewise, confectionary compositions typically have a relatively high concentration of active ingredient to enable sufficient dispersion as they dissolve or are masticated.

In various embodiments of the present invention, active compound(s) from magnolia is present in the oral care composition in a concentration of about 0.001 to about 10% by weight. In one embodiment, it is present in the oral care composition in a concentration of about 0.01 to about 3%. In other embodiments, it is present at less than 1%, for example the extract is at a concentration of about 0.01 to about 1%. In one preferred embodiment, the compound is present in the oral care composition at a concentration of about 0.3%.

In certain embodiments of the present invention, additional antibacterial ingredients may be included in the oral care compositions. If added, the antibacterial active ingredients it is desirable that the additive does not substantially detract from the efficacy and bioavailability of the tartar control agents or the active compound of the extract. Preferably, the additional antibacterial active ingredients are nonionic.

Suitable additional antibacterial agents for use in the present invention include other known antibacterial botanical extracts or active compounds isolated from such extracts, such as those isolated from green or oolong tea, gold thread, cranberry and other Ericaceae family plants, honeysuckle, grape seed, myrobalan, rosemary, east Indian walnut, neem, niruri, pine bark, compounds from camellia sinensis, catechin, epocatechin, epigallocatechin, epicatchin gallate, gallocatechin, epigallocatechin, extracts from the plant families Annonaceae, Berberidaceae, Menispermaceae, Papaveraceae, Ranunculaceae, Rutaceae, Zingiberaceae, Nadina, Mahonia, Thalictrum spp, berberine, Lonicera ceprifolium extracts, chlorogenic acid, lutenolin flavanoids. Other suitable additional antibacterial agents include compounds from the Ericaceae, such as those disclosed in U.S. Pat. No. 5,980,869, the contents of which are incorporated herein by reference. In certain embodiments, extracts from plants in the Vaccinium genus are useful as adding antibacterial active agents, such as cranberry (Vaccinium macrocarpon).

Extracts suitable for use in the present invention can be obtained from any part of the plant including the leaf, stem, bark, pulp, seed, flesh, juice, root and mixtures thereof. It is preferred that the extract is obtained from the leaf, pulp and seed, more preferably from the leaf, flower or bark.

In various embodiments of the present invention, the oral composition comprises an anticalculus system that prevents calculus formation on the surface of the teeth in a subject. One type of useful anticalculus ingredient is a linear molecularly dehydrated polyphosphate salt. Polyphosphate salts are generally employed in the form of their wholly or partially neutralized water soluble alkali metal (e.g., potassium, sodium or ammonium salts, and any mixtures thereof).

The present invention includes an effective combination of anticalculus ingredients comprising polyphosphate compounds. In particular, a first preferred active anticalculus ingredient is sodium tripolyphsophate (STPP). A second preferred active anticalculus ingredient is tetrasodium pyrophosphate (TSPP). In various embodiments, the combination of the STPP and TSPP increases the effectiveness of the anticalculus system, without a concomitant increase in required dosage level or rate of administration, so that lower quantities of overall tartar control system can be administered, yet still achieve the desired therapeutic effect. In certain embodiments of the present invention, the ratio of the first anticalculus active ingredient, tetrasodium pyrophosphate, to the second anticalculus active ingredient, ranges about 1:2 to about 1:4. In one preferred embodiment of the present invention, the first anticalculus active ingredient, tetrasodium pyrophosphate, is present in the oral care composition about 1 to about 2.5% and the second anticalculus active ingredient, sodium tripolyphosphate is present about 1 to about 10%. However, as discussed in the context of the magnolia extract above, such concentrations of active ingredients may vary based upon the form of the oral composition, where they are generally higher for confectioneries and dentifrices and generally lower for mouth washes or rinses.

In another embodiment of the present invention, the ratio of the first anticalculus active ingredient, tetrasodium pyrophosphate, to the second anticalculus active ingredient, ranges about 1:3 to about 1:4. In an embodiment, the first anticalculus active ingredient, TSPP is present about 1 to about 2.5% and the second anticalculus active ingredient, STPP is present about 3 to about 7%. In another embodiment, the ratio of TSPP to STPP is about 2:3. For example, the anticalculus system comprises the first anticalculus active ingredient TSPP present at about 2% and the second anticalculus active ingredient STPP present at about 3% of the oral care composition.

Various embodiments of the present invention include an anticalculus system that further comprises a synthetic anionic linear polycarboxylate polymer. The -anionic linear polycarboxylate is generally synthesized by using an olefinically or ethylenically unsaturated carboxylic acid that contains an activated carbon-to-carbon olefinic double bond and at least one carboxyl group. The acid contains an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrilacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other olefinic monomers copolymerizable with such carboxylic monomers include vinyl acetate, vinyl chloride, dimethyl maleate and the like. The synthetic anionic linear polymeric polycarboxylate component is mainly a hydrocarbon with optional halogen and O-containing substituents and linkages as present in for example ester, ether and OH groups. The copolymers preferably contain sufficient carboxylic salt groups for water-solubility. The terms “synthetic” and “linear” do not include known thickening or gelling agents comprising carboxymethylcellulose and other derivatives of cellulose and natural gums, nor Carbopols having reduced solubility due to cross-linkages. Also excluded are the zinc, magnesium and similar metal complexes of these polymeric polycarboxylates.

Preferred are 1:4 to 4:1 copolymers of maleic anhydride or acid with another polymerizable ethylenically unsaturated monomer, preferably methyl vinyl ether (methoxyethylene) having a molecular weight (M.W.) of about 30,000 to about 1,000,000. These copolymers are commercially available, for example as Gantrez AN-139 (M.W. 1,000,000), AN-119 (M.W. 200,000) and S-97 Solution (M.W. 1,500,000), from ISP Corporation.

Thus, in certain embodiments of the present invention, the anticalculus system further comprises a synthetic anionic polycarboxylate polymer, in addition to the tetrasodium pyrophosphate and the sodium tripolyphosphate. In one embodiment, the synthetic anionic polycarboxylate is present about 0.1 to about 5%. In another embodiment, the synthetic anionic polycarboxylate is present about 0.5 to about 1.5%, most preferably at about 1% of the oral care composition. In one embodiment according to the present invention, the anticalculus system comprises a copolymer of maleic anhydride and methyl vinyl ether, such as for example, the Gantrez S-97 product discussed above.

Thus, in some embodiments, the oral composition comprises a ratio of the first anticalculus active ingredient, TSPP to the second anticalculus active ingredient STPP to the synthetic anionic polycarboxylate, which ranges about 5:10:1 to about 5:20:10 (or 1:4:2). In one embodiment, the anticalculus system of the oral care composition comprises TSPP at about 1%, STPP at about 7%, and a copolymer of maleic anhydride and methyl vinyl ether at about 1% (or a ratio of 1:7: 1). In certain embodiments of the present invention, the anticalculus system may exclude other tartar control active ingredients, and may consist essentially of: tetrasodium pyrophosphate (TSPP) and sodium tripolyphosphate (STPP), and a copolymer of maleic anhydride and methyl vinyl ether. In another embodiment, the anticalculus system consists essentially of a ratio of TSPP to STPP to a copolymer of maleic anyhydride and methyl vinyl ether about 1:2:1 to about 2:7:1. For example, in one embodiment, the anticalculus system consists essentially of TSPP present at about 0.5 to about 2.5%, STPP present at about 1 to about 10%, and a copolymer of maleic anhydride and methyl vinyl ether present at about 0.5 to about 1.5%

In various embodiments, the composition includes a carrier. The carrier may comprise a water-phase. As recognized by one of skill in the art, the oral compositions of the present invention optionally include other materials, such as for example, anticaries agents, densensitizing agents, viscosity modifiers, diluents, surface active agents, such as surfactants, emulsifiers, and foam modulators, pH modifying agents, abrasives, humectants, mouth feel agents, sweetening agents, flavor agents, colorants, preservatives and combinations thereof. It is understood that while general attributes of each of the above categories of materials may differ, there may be some common attributes and any given material may serve multiple purposes within two or more of such categories of materials. Preferably, such carrier materials are selected for compatibility with the antibacterial magnolia active ingredient and the anticalculus tartar control system, as well as with other ingredients of the composition.

The term “mouthrinse” in the present invention refers to oral compositions that are substantially liquid in character, such as a mouth wash, spray, or rinse. In such a preparation the orally acceptable carrier typically has an aqueous phase comprising a water and alcohol mixture. Further, in various embodiments, the oral carrier has a humectant and surfactant as described below. Generally, the weight ratio of water to alcohol is in the range of about 1:1 to about 20:1, preferably about 3:1 to 10:1, and more preferably about 4:1 to about 6:1. The total amount of water-alcohol mixture in this type of preparation is typically in the range of about 70 to about 99.9% of the preparation. In various embodiments, the alcohol is typically ethanol or isopropanol.

The pH of such liquid and other preparations of the invention is generally in the range of about 4.5 to about 9. The pH can be controlled with acid (e.g. citric acid or benzoic acid) or base (e.g., sodium hydroxide) or buffered (with sodium citrate, benzoate, carbonate, or bicarbonate, disodium hydrogen phosphate, or sodium dihydrogen phosphate, for example).

In various embodiments, the aqueous oral composition (e.g., mouthrinse) contains a humectant. The humectant is generally a mixture of humectants, such as glycerin and sorbitol, and a polyhydric alcohol such as propylene glycol, butylene glycol, hexylene glycol, polyethylene glycol. The humectant content is in the range of about 5 to abut 40% and preferably about 10 to about 30%. Surfactants useful in the present embodiment include anionic, nonionic, and zwitterionic surfactants. The surfactant is present in the aqueous oral compositions of the present invention range about 0.1% to about 5% preferably about 0.6% to about 2.0%.

The term “confectionery composition” as used herein includes chewing gums, and orally soluble tablets, beads and lozenges. Saliva dissolves the lozenge or chewable gum product, and promotes prolonged contact with oral surfaces so that the delivery of the antibacterial agent and the anticalculus system in a lozenge tablet, bead or chewing gum form ensures that an adequate dosage of the active ingredients are delivered to the oral surface when the product is used.

In the present embodiment, the orally acceptable carrier is in the form of a lozenge, bead, tablet or chewing gum or other similar solid delivery system. Such delivery systems are well known to one of skill the art, and generally entail stirring the active antibacterial and anticalculus agents into a warm base with flavor, and non-cariogenic sweeteners.

The orally acceptable vehicle or carrier in a lozenge bead or tablet is a non-cariogenic, solid water-soluble polyhydric alcohol (polyol) such as mannitol, xylitol, sorbitol, malitol, hydrogenated starch hydrozylate, hydrogenated glucose, hydrogenated disaccharides or hydrogenated polysaccharides, in an amount of about 85 to about 95% of the total composition. Emulsifiers such as glycerin, and tableting lubricants, in minor amounts of about 0.1 to 5%, may be incorporated into the tablet, bead or lozenge formulation to facilitate the preparation of the tablet beads and lozenges. Suitable lubricants include vegetable oils such as coconut oil, magnesium stearate, aluminum stearate, talc and starch. Suitable noncariogenic gums include kappa carrageenan, carboxymethyl cellulose, hydroxyethyl cellulose and the like.

The lozenge, bead or tablet may optionally be coated with a coating material such as waxes, shellac, carboxymethyl cellulose, polyethylene/maleic anhydride copolymer or kappacarrageenan to further increase the time it takes the tablet or lozenge to dissolve in the mouth. The uncoated tablet or lozenge is slow dissolving, providing a sustained release rate of active ingredients of about 3 to 5 minutes. Accordingly, the solid dose tablet, bead and lozenge compositions of this embodiment affords a relatively longer time period of contact of the teeth in the oral cavity with the antibacterial and anticalculus active ingredients of the present invention.

The chewing gum of the present invention is preferably a sugarless chewing gum containing the antibacterial and anticalculus compounds. Chewing gum formulations typically contain, in addition to, a chewing gum base, one or more plasticizing agents, at least one sweetening agent and at least one flavoring agent.

Gum base materials suitable for use in the practice of this invention are well known in the art and include natural or synthetic gum bases or mixtures thereof. Representative natural gums or elastomers include chicle, natural rubber, jelutong, balata, guttapercha, lechi caspi, sorva, guttakay, crown gum, perillo, or mixtures thereof. Representative synthetic gums or elastomers include butadiene-styrene copolymers, polyisobutylene and isobutylene-isoprene copolymers. The gum base is incorporated in the chewing gum product at a concentration of about 10 to about 40% and preferably about 20 to about 35%.

Plasticizing/softening agents commonly used in chewing gum compositions are suitable for use in this invention, including gelatin, waxes and mixtures thereof in amounts of about 0.1 to about 5%. The sweetening agent ingredient used in the practice of this invention may be selected from a wide range of materials, and include the same artificial and polyol sweeteners used for the preparation of tablets, beads and lozenges. Polyol sweeteners such as sorbitol and malitol are present in the chewing gum composition of the present invention in amounts of about 40 to about 80% and preferably about 50 to about 75%. The artificial sweetener is present in the chewing gum composition of the present invention in amounts of about 0.1 to about 2% and preferably about 0.3 to about 1%.

In certain other desirable forms of this invention, the oral composition may be a dentifrice. As referred to herein, a “dentifrice” is a composition that is intended for cleaning a hard surface within the oral cavity. Such dentifrices include toothpowder, a dental tablet, toothpaste (dental cream), or gel. In a toothpaste dentifrice, the orally acceptable carrier may comprise water and humectant typically in an amount ranging about 10% to about 80% of the oral composition.

In various embodiments of the present invention, glycerin, propylene glycol, sorbitol, polypropylene glycol and/or polyethylene glycol (e.g., 400-600) are suitable humectants/carriers. Also advantageous are liquid mixtures of water, glycerin and sorbitol. In certain embodiments where the carrier is a clear gel and where the refractive index is an important consideration, the composition comprises about 3 to about 30% of water, 0 to about 70% of glycerin and about 20-80% of sorbitol.

The oral composition may contain other conventional ingredients, such as humectants, thickeners, surface active agents, flavorants, colorants, abrasives, whitening agents, polishing materials, water, alcohol, active pharmaceutical agents, preservatives, and sweeteners.

In various embodiments of the present invention, water is also present in the oral composition, as referred to above. Water employed in the preparation of commercially suitable toothpastes, gels, and mouthwashes should preferably be deionized and free of organic impurities. Water generally comprises about 10% to 50%, preferably about 20% to 40%, of the toothpaste compositions herein. The water is free water which is added, plus that which is introduced with other materials for example, such as that added with sorbitol.

In various embodiments, the present invention provides a method of treating plaque and calculus on an oral surface (tooth surface) of a mammalian subject, where the method comprises first preparing an oral care composition. In certain embodiments, the oral care composition comprises an orally acceptable carrier, as well as a safe and effective amount of an antibacterial ingredient comprising an extract of magnolia. The oral care composition also comprises a safe and effective amount of an anticalculus system comprising tetrasodium pyrophosphate (TSPP) and sodium tripolyphosphate (STPP). In one embodiment, the TSPP is present about 0.5 to about 2.5% of the composition and the STPP is present about 1 to about 10% of the oral care composition. The oral care composition is contacted with the oral surface of the mammalian subject to thereby kill bacteria and reduce plaque formation, calculus formation, and reduce inflammation, in a highly efficacious manner, without any negative interaction between the anticalculus system, the antibacterial system, or the orally acceptable carrier. In various embodiments, it is preferred that the oral care composition is applied and contacted with the oral surface at least one time daily and repeated for multiple days.

Thus, in various embodiments of the present invention, a dentifrice, confectionary, or mouthwash prepared in accordance with the present invention is preferably applied regularly to an oral surface, preferably on a daily basis, at least one time daily for multiple days, but alternately every second or third day. Most preferably the oral composition is applied to the oral surfaces from 1 to 3 times daily, at a pH of about 4.5 to about 9, generally about 5.5 to about 8, preferably about 6 to 8, for at least 2 weeks up to 8 weeks or more up to lifetime.

The oral compositions of the present invention may be prepared by suitably mixing the ingredients. For instance, in the preparation of a mouthrinse, the antibacterial active ingredient comprising magnolia extract and the anticalculus system are dispersed in a mixture of ingredients, e.g., alcohol, humectants, surfactants, and flavor. The ingredients are then mixed under vacuum for about 15-30 minutes. The resulting rinse product is then packaged. Dentifrices are prepared similarly, additional thickener and abrasives agents being included in the last step.

The antiplaque, anticalculus, and antigingivitis oral composition of this invention can be incorporated into confectionary and tropes. Such methods of forming confectionary (e.g., gum) or tropes (e.g., lozenges) are well known by one of skill in the art, and can be prepared by stirring into a warm gum base or coating the outer surface of a gum base (for example, jelutone, rubber latex, vinylite resins, inter alia), desirably with conventional plasticizers or softeners, sugar or other sweeteners or carbohydrates such as glucose, sorbitol and the like.

The present invention is further illustrated through the following non-limiting examples.

### EXAMPLE I

A dentifrice formulation is prepared containing 0.3% solution of magnolia extract extracted with HFA-13A containing approximately 15% honokiol and 37% magnolol and a tartar control system containing tetrasodium pyrophosphate (TSPP), sodium tripolyphosphate (STPP) and a copolymer of maleic anhydride and methyl vinyl ether (GANTREZ® S97 liquid). Specifically, a dentifrice composition having the ingredients listed in Table I is prepared by the following method. The humectants e.g., glycerin and sorbitol, are dispersed in water in a conventional mixer under agitation. The flavor oil is weighed out and magnolia is then added to the favor oil. The flavor oil and magnolia mixture is added with sodium lauryl sulfate (SLS) into the mixer. Then organic thickeners, such as carageenan, any salts, such as sodium fluoride anticaries agents, and the anticalculus active ingredient system of tetrasodium pyrophosphate (TSPP), sodium tripolyphosphate (STPP) and GANTREZ® liquid; as well as sweeteners (saccharin) are added. The resultant mixture is agitated until a homogeneous gel phase is formed. A pigment such as TiO₂ is added into the gel phase, and any acid or base (e.g., NaOH) required to adjust the pH to 6 to 7. These ingredients axe mixed until a homogenous phase is obtained. The mixture is then transferred to a high-speed vacuum mixer; where silica abrasive SYLODENT® XWA 650 and silica thickener ZEODENT® 165 are added. The mixture is then mixed at high speed for from 5 to 30 minutes, under vacuum of about 20 to 50 mm of Hg, preferably about 30 mm Hg. The resultant product is a homogeneous, semi-solid, extrudable paste or gel product.

TABLE I

| Ingredient | Weight % |
|---|---|
| Magnolia Cortex Extract | 0.3 |
| TSPP | 1.0 |
| STPP | 7.0 |
| GANTREZ ® S97- liquid solution (13% in H₂O) | 7.7 |
| Sorbitol (70% in H₂O) | 26.7 |
| Glycerin | 12.0 |
| Sodium fluoride | 0.3 |
| Sodium saccharin | 0.5 |
| Sodium hydroxide (50% in H₂O) | 2.0 |
| CMC 2000S | 0.8 |
| Carrageenan (LB 9505) | 0.4 |
| SYLODENT ® 783 | 11.0 |
| SYLODENT ® XWA 650 | 10.0 |
| ZEODENT ® 165 | 3.5 |
| Sodium lauryl sulfate (30% conc.) | 4.0 |
| TiO₂ coated Mica | 0.1 |
| Flavor (89-332) | 1.0 |
| Blue Color Solution | 0.05 |
| Water | Q.S. |

### EXAMPLE II

A dentifrice formulation containing the oral composition of the present invention is formed by the method described above in Example I. However, in the present example, the anticalculus system is comprised of TSPP and STPP and no Gantrez is added to oral composition.

TABLE II

| Ingredient | Weight % |
|---|---|
| Magnolia Cortex Extract | 0.3 |
| TSPP | 2.0 |
| STPP | 3.0 |
| Sorbitol (70% in H₂O) | 26.7 |
| Glycerin | 12.0 |
| Sodium fluoride | 0.3 |
| Sodium saccharin | 0.5 |
| Sodium hydroxide (50% in H₂O) | 2.0 |
| CMC 2000S | 0.8 |
| Carrageenan (LB 9505) | 0.4 |
| SYLODENT ® 783 | 11.0 |
| SYLODENT ® XWA 650 | 10.0 |
| ZEODENT ® 165 | 3.5 |
| Sodium lauryl sulfate (30% conc.) | 4.0 |
| TiO₂ coated Mica | 0.1 |
| Flavor (89-332) | 1.0 |
| Blue Color Solution | 0.05 |
| Water | Q.S. |

The examples and other embodiments described herein are exemplary and not intended to be limiting in describing the full scope of compositions and methods of this invention. Equivalent changes, modifications and variations of specific embodiments, materials, compositions and methods may be made within the scope of the present invention, with substantially similar results.

We claim:

1. An oral composition comprising:
(a) an active compound from an extract of magnolia; and
(b) an anticalculus system comprising tetrasodium pyrophosphate (TSPP) and sodium tripolyphosphate (STPP), wherein said TSPP is present about 0.5 to about 2.5% and said STPP is present in an amount of about 1 to about 10% by weight of the composition.

2. The oral composition according to claim 1, wherein the active compound is present at a concentration of about 0.001 to about 10%.

3. The oral composition according to claim 1, wherein the active compound is present at a concentration of about 0.01 to about 3%.

4. The oral composition according to claim 1, wherein the active compound is present at a concentration of less than about 1%.

5. The oral composition of claim 1, wherein the anticalculus system further comprises an anionic polycarboxylate.

6. The oral composition according to claim 5, wherein the anionic polycarboxylate is a copolymer of maleic anhydride and methyl vinyl ether.

7. The oral composition according to claim 1, wherein the anticalculus system comprises TSPP at about 0.5 to about 1.5%, STPP at about 6.5 to about 7.5%.

8. The oral composition according to claim 1, wherein the anticalculus system comprises TSPP at about 0.5 to about 2.5% and STPP at about 1 to about 3%.

9. The oral composition according to claim 1, wherein the active compound is selected from magnolol, honokiol, tetrahydromanolol, and tetrahydrohonokiol.

10. The oral composition according to claim 1, wherein the active compound is magnolol in a concentration of about 2% to about 95%.

11. An oral composition according to claim 1, wherein the active compound is magnolol in a concentration of about 5% to about 50%.

12. The oral care composition according to claim 1, wherein the oral care composition is in a form selected from the group consisting of: a mouthrinse, a confectionary, a dentifrice, and a dissolvable tablet.

13. The oral composition according to claim 1, wherein the active compound is honokiol in a concentration of about 2% to about 95%.

14. The oral composition according to claim 1, wherein the active compound is honokiol in a concentration of about 5 to about 50%.

15. The oral care composition according to claim 1, wherein the active compound is extracted from magnolia by supercritical fluid extraction.

16. A method preventing formation of plaque and tartar on an oral surface in a mammalian subject that comprises:
(a) applying an oral care composition according to claim 1 to the tooth surface; and
(b) repeating (a) for multiple days to minimize formation of plaque and tartar the oral surface.

17. A method of promoting or maintaining systemic health in a human comprising applying an effective amount of an oral care composition of claim 1 to the oral cavity.

18. The method of claim 17, wherein the composition further comprises an anionic polycarboxylate.

19. A stable efficacious antiplaque, anticalculus, and antigingivitis oral composition comprising:
(a) a safe and effective amount of an antibacterial ingredient comprising an extract of magnolia at a concentration of about 0.001 to about 10% of the composition;
(b) a safe and effective amount of an anticalculus system consisting essentially of tetrasodium pyrophosphate (TSPP), sodium tripolyphosphate (STPP), and a copolymer of maleic anhydride and methyl vinyl ether, wherein said TSPP is present about 0.5 to about 2.5%, said STPP is present about 1 to about 10%, and said copolymer of maleic anhydride and methyl vinyl ether is present about 0.5 to about 1.5% based on the total weight of the composition; and
(c) an orally acceptable carrier.

20. A method of treating plaque and calculus on an oral surface of a mammalian subject, the method comprising:
preparing an oral care composition comprising an orally acceptable carrier; a safe and effective amount of an antibacterial ingredient comprising at least one active compound from an extract of magnolia; a safe and effective amount of an anticalculus system comprising tetrasodium pyrophosphate (TSPP) and sodium tripolyphosphate (STPP), wherein said TSPP is present about 0.5 to about 2.5% and said STPP is present about 1 to about 10% based on the total weight of said composition; and
contacting said oral care composition with the oral.

21. A method according to claim 20, wherein said oral care composition further comprises a copolymer of maleic anhydride and methyl vinyl ether at about 0.5 to about 1.5%.

22. A method according to claim 20, wherein said contacting of said oral care composition to the oral surface is repeated at least one time daily for multiple days.

23. A stable antiplaque, anticalculus, and antigingivitis oral composition comprising:
(a) a safe and effective amount of an antibacterial ingredient comprising at least one active compound from an extract of magnolia;
(b) a safe and effective amount of an anticalculus system comprising tetrasodium pyrophosphate (TSPP) and sodium tripolyphosphate (STPP), wherein a ratio of said TSPP to said STPP ranges about 1:2 to about 1:4 in said anticalculus system; and
(c) an orally acceptable carrier.

24. An oral composition according to claim 23, wherein said at least one active compound from said magnolia extract is present in the oral composition at a concentration of about 0.001 to about 10%.

25. An oral composition according to claim 23, wherein said at least one active compound from said magnolia extract is present in the oral composition at a concentration of less than about 1%.

26. An oral composition according to claim 23, wherein said anticalculus system further comprises a copolymer of maleic anhydride and methyl vinyl ether, and a ratio of said TSPP to said STPP to said copolymer ranges about 5:10:1 to about 1:4:2 in said anticalculus system.

27. An oral composition according to claim 23, wherein said anticalculus system further comprises a copolymer of maleic anhydride and methyl vinyl ether, and a ratio of said TSPP to said STPP to said copolymer ranges about 1:2:1 to about 2:7:1 in said anticalculus system.
